(19) United States
(12) Patent Application Publication
(10) Pub. No.: US 2005/0163847 A1
(43) Pub. Date: Jul. 28, 2005

(54) Pharmaceutical formulations containing a non-steroidal antiinflammatory drug and an antiulcerative drug

(75) Inventors: Xiu Xiu Cheng, Weston, FL (US); Avinash Nangia, Lincoln, RI (US); Dacheng Tian, Miramar, FL (US)

Correspondence Address: DAVIDSON, DAVIDSON & KAPPEL, LLC, 485 SEVENTH AVENUE, 14TH FLOOR, NEW YORK, NY, 10018, US

(73) Assignee: Andrx Pharmaceuticals, LLC, Plantation, FL (US)

(21) Appl. No.: 11/039,489

(22) Filed: Jan. 20, 2005

Related U.S. Application Data
(60) Provisional application No. 60/537,862, filed on Jan. 21, 2004.

(51) Int. Cl. A61K 31/60; A61K 31/405; A61K 31/192; A61K 9/24

(52) U.S. Cl. 424/472; 514/165; 514/420; 514/569; 514/570

(57) ABSTRACT

Disclosed is a pharmaceutical dosage form including a therapeutically effective amount of an NSAID and an antiulcerative agent.

### FIELD OF THE INVENTION

The present invention is related to a pharmaceutical formulation comprising a non-steroidal anti-inflammatory drug (“NSAID”) and an antiulcerative drug in a single oral pharmaceutical dosage form.

### BACKGROUND OF THE INVENTION

Although NSAIDs are often used for their antiinflammatory, analgesic, and/or antipyretic effects, it is well known that NSAIDs have the potential to cause gastrointestinal (GI) bleeding through a variety of mechanisms related to their topical and systemic effects. The GI bleeding may depend on the length of the treatment and on the particular drug. This problem is important in cases where the therapy must be continued for a long period of time. For example, osteoarthritis and rheumatoid arthritis in the elderly is often treated with long-term NSAID therapy, as chronic treatment is needed to control pain and inflammation and to improve quality of life.

Additionally it is well known that because of their side-effects on the GI tract, NSAIDs are invariably administered after meals or, generally, when the stomach is not empty. This pharmacological principle is confirmed by the recommendations found in the labeling of these medications. The basic idea set forth is that the effects of the hypersecretion of hydrochloric acid provoked by the administration of NSAIDs may be, at least partially, counteracted by the presence of food. Patients who have an ulcer or who are susceptible to developing ulcers are commonly advised to avoid taking NSAIDs for pain, inflammation, and/or fever.

There is a continuing need for analgesic medications to provide high efficacy pain relief while reducing the possibility of undesirable effects. Non-steroidal anti-inflammatory drugs, including compounds such as ibuprofen, ketoprofen and diclofenac, have anti-inflammatory actions and are effective on pain associated with the release of prostaglandins and other mediators of inflammation. For example, diclofenac is considered to be extremely potent and effective as an analgesic and anti-inflammatory agent. Diclofenac is approved in the United States for the long-term symptomatic treatment of rheumatoid arthritis, osteoarthritis and alkylosing spondylitis. It is also considered to be useful for the short-term treatment of acute musculoskeletal injury, acute painful shoulder, postoperative pain and dysmenorrhea. However, NSAIDs such as diclofenac produce side effects in about 20% of patients that require cessation of medication. Side effects include, for example, gastrointestinal bleeding and the abnormal elevation of liver enzymes.

Non-steroidal anti-inflammatory drugs (NSAIDs) exert most of their anti-inflammatory, analgesic and antipyretic activity and inhibit hormone-induced uterine contractions and certain types of cancer growth through inhibition of prostaglandin G/H synthase, also known as cyclooxygenase. Inhibition of COX-1 causes a number of side effects including inhibition of platelet aggregation associated with disorders of coagulation, and gastrointestinal toxicity with the possibility of ulcerations and of hemorrhage. It is believed that the gastrointestinal toxicity is due to a decrease in the biosynthesis of prostaglandins which are cytoprotective of the gastric mucosa.

A high incidence of side effects has historically been associated with chronic use of classic cyclooxygenase inhibitors, all of which are about equipotent for COX-1 or COX-2, or which are COX-1-selective. While renal toxicity occurs, it usually becomes evident in patients who already exhibit renal insufficiency (D. Kleinknecht, Sem. Nephrol. 15: 228, 1995). By far, the most prevalent and morbid toxicity is gastrointestinal. Even with relatively nontoxic drugs such as piroxicam, up to 4% of patients experience gross bleeding and ulceration (M. J. S. Langman et al, Lancet 343: 1075, 1994). In the United States, it is estimated that some 2000 patients with rheumatoid arthritis and 20,000 patients with osteoarthritis die each year due to gastrointestinal side effects related to the use of COX inhibitors. In the UK, about 30% of the annual 4000 peptic ulcer-related deaths are attributable to COX inhibitors (Scrip 2162, p. 17). COX inhibitors cause gastrointestinal and renal toxicity due to the inhibition of synthesis of homeostatic prostaglandins responsible for epithelial mucus production and renal blood flow, respectively.

NSAID therapy inhibits prostaglandin synthesis and causes a deficiency of prostaglandins within the gastric and duodenal mucosa which may lead to reduced bicarbonate and mucus secretion and may contribute to mucosal damage.

Measures which can be taken to decrease GI side affects associated with NSAID therapy is to coadminister a prostaglandin analogue, e.g. misoprostol, an H2 blocker, e.g. ranitidine, or a proton pump inhibitor, e.g. omeprazole, with the NSAID.

Prostaglandin replacement therapy has been demonstrated to prevent NSAID induced ulcers. Prostaglandin analogues useful in such therapy are described in U.S. Pat. Nos. 3,965,143, 4,060,691, 4,271,314 and 4,683,328, which are hereby incorporated by reference. A commercially available prostaglandin analogue is misoprostol, which is a synthetic prostaglandin E1 analog with gastric antisecretory and mucosal protective properties.

U.S. Pat. No. 5,601,843 describes a formulation which comprises a core comprising an NSAID selected from diclofenac or piroxicam, and a mantle coating consisting of a prostaglandin enveloping the core.

Proton pump inhibitors are antisecretory agents that suppress gastric acid secretion by the inhibition of the H⁺, K⁺—ATPase enzyme system at the secretory surface of the gastric parietal cell.

H₂ inhibitors block the action of histamine on stomach cells, and reduce stomach acid production and are useful in promoting healing of stomach and duodenal ulcers, and in reducing ulcer pain. H₂ inhibitors have been effective in preventing ulcer recurrence when given in low doses for prolonged periods of time.

There exists a need in the art for a combination formulation which includes an NSAID with a second agent to reduce the occurrence of gastro-intestinal side effects associated with NSAID treatment.

### SUMMARY OF THE INVENTION

It is an object of certain embodiments of the present invention to provide a method for the treatment of pain, inflammation, and/or fever with the use of an NSAID formulation with decreased gastro-intestinal side effects typically associated with NSAID therapy.

It is a further object of certain embodiments of the present invention to provide a solid oral dosage form which decreases the risk of the development and/or exacerbation of ulcers which may occur during NSAID therapy.

It is a further object of certain embodiments of the present invention to provide a solid oral dosage form which promotes patient compliance and thereby increases the efficacy of NSAID treatment in patients who are being chronically treated with NSAIDs.

It is a further object of certain embodiments of the present invention to provide a solid oral dosage form for the treatment of a human patient on NSAID therapy or about to begin NSAID therapy, which decreases or minimize gastrointestinal side-effects.

In view of the above-mentioned objects and others, the invention is directed in part to an oral solid dosage form comprising a therapeutically effective amount of an NSAID and an antiulcerative compound in an amount effective to decrease or prevent gastrointestinal side effects normally associated with the NSAID treatment.

In certain embodiments, the present invention is directed to a solid oral dosage form comprising a NSAID portion and an antiulcerative portion, wherein the antiulcerative portion partially surrounds the NSAID portion. The antiulcerative portion may be applied or compressed around or onto the NSAID portion.

In certain embodiments, the present invention is directed to a solid oral dosage form comprising an NSAID portion having at least one internal hole extending through the NSAID portion; and a coating portion comprising an antiulcerative compound. In certain preferred embodiments, the internal hole extends through the center of the NSAID portion, causing the NSAID portion to have a “donut-like” configuration. In certain embodiments, the internal hole is at least partially filled with the antiulcerative compound forming an antiulcerative core. In other embodiments, the internal hole is at least partially filled with the antiulcerative compounds and at least a portion of the outer surface of the NSAID portion is covered by the antiulcerative compound.

In certain embodiments, the present invention is directed to a solid oral dosage form comprising a) an NSAID portion comprising an NSAID; the NSAID portion having a top surface, a bottom surface, and an internal hole extending from the top surface of the NSAID portion to the bottom surface of the NSAID portion; and b) a coating comprising an antiulcerative compound wherein the coating fills the internal hole to form an antiulcerative compound core. Preferably, the coating also covers at least a portion of the NSAID formulation.

In certain embodiments, in addition to the NSAID portion having a top surface, a bottom surface, and an internal hole as described above, the NSAID portion may also have an inner circumferential surface formed by the internal hole within the NSAID portion and an outer circumferential surface.

In certain embodiments of the invention wherein the NSAID portion has an internal hole, the coating comprising an antiulcerative compound may completely surround the NSAID portion such that the dosage form has a hollow core (e.g., the internal circumferential surface is also coated) or such that the coating fills the hole and no hollow core is present. In such an embodiment wherein there is a hollow core, the final product has the appearance of a circular ring or donut-like configuration.

In certain embodiments, the present invention is directed to a method of preparing a solid oral dosage form comprising compression coating an NSAID portion comprising an NSAID and one or more pharmaceutically acceptable excipients with a mixture of an antiulcerative compound and one or more pharmaceutically acceptable excipients to form a solid oral dosage form comprising a NSAID portion and an antiulcerative portion, wherein the antiulcerative portion partially surrounds the NSAID portion.

In preferred embodiments of the invention, the antiulcerative compound is a prostaglandin, most preferably misoprostol and the NSAID is diclofenac or a salt thereof (e.g., the sodium or potassium salt). Preferably the diclofenac is in tablet form and the misoprostol is coated by compression coating.

The solid oral dosage form can be prepared in accordance with known procedures in the art and can be an immediate release, controlled release, delayed release or sustained release formulation. In certain embodiments, all or part of the NSAID is in controlled release, delayed release or sustained release form. In certain embodiments, all or part of the antiulcerative compound is in controlled release, delayed release or sustained release form. In certain embodiments, both the NSAID and the antiulcerative compound are all or partially in controlled release, delayed release or sustained release form.

The formulation comprising the NSAID is preferably layered with a material suitable to prevent contact of said NSAID with acidic gastric juice after oral administration, such as an enteric coating. In certain embodiments, the enteric coating covers the formulation prior to coating with the antiulcerative compound, and provides for a barrier layer between the NSAID portion and the antiulcerative portion.

In certain embodiments, the present invention is directed to a method of preparing a solid oral dosage form comprising a) mixing an NSAID with one or more pharmaceutically acceptable excipients; b) compressing the mixture to form a NSAID portion comprising an NSAID and at least one internal hole extending through the NSAID portion; or optionally compressing the mixture and boring the internal hole after compression; c) applying or compressing a coating portion comprising an antiulcerative compound into the internal hole and optionally on at least a portion of the outer surface of the NSAID portion.

In certain preferred embodiments, the coating is applied onto the NSAID portion by compression coating.

In certain embodiments of the present invention the internal hole does not extend all the way through the NSAID portion but forms a cavity or recess in the NSAID portion which may thereafter be filed with the antiulcerative agent or coating comprising the antiulcerative agent.

In certain embodiments, the present invention is further directed to a solid oral dosage form comprising a therapeutically effective amount of an NSAID contained in a plurality of multiparticulates; the NSAID multiparticulates dispersed in a matrix comprising a therapeutically effective amount of an antiulcerative compound.

In certain embodiments, the present invention is further directed to a solid oral dosage form comprising a therapeutically effective amount of an NSAID coated onto a plurality of pharmaceutically acceptable inert beads and overcoated with a delayed release coating (e.g. an enteric coating), wherein the delayed release NSAID beads dispersed in a matrix comprising a therapeutically effective amount of an antiulcerative compound.

In certain embodiments, the present invention is directed to a method of preparing a solid oral dosage form comprising a) coating an NSAID onto a plurality of pharmaceutically acceptable inert beads; b) overcoating the beads with a delayed release coating; c) blending the beads with a mixture comprising an antiulcerative compound and at least one pharmaceutically acceptable excipient; and d) compressing a sufficient amount of the blend into a dosage form to contain a therapeutically effective amount of the NSAID and the antiulcerative compound, wherein said NSAID beads are dispersed in the matrix material; or optionally incorporating a sufficient amount of the blend into a capsule.

In certain embodiments the invention is further directed to a method of treating a human patient in need of antiinflammatory, analgesic and/or antipyretic therapy, comprising orally administering to the patient a solid oral dosage form of the present invention, the dosage form comprising a therapeutically effective amount of an NSAID and an amount of an antiulcerative compound effective to prevent and/or reduce gastrointestinal side effects of the NSAID.

Preferably the inventive formulations and methods described herein promote patient compliance and thereby increase efficacy of NSAID treatment in patients who are being chronically treated with NSAIDs. In other words, the inventive formulations increase the likelihood that a patient on NSAID therapy who is noncompliant due to gastrointestinal side effects, or who forgets or refuses to take both medications separately will be more accepting of a single composition combining the NSAID and antiulcerative compound, particularly due to the prevention and/or treatment of gastrointestinal side effects. For purposes of the present invention, all references to antiulcerative compounds (e.g., prostaglandins) and NSAIDs include their single enantiomers, isomers and their pharmaceutically acceptable salts (e.g., diclofenac sodium or diclofenac potassium).

For purposes of the present invention, the term “partially surrounds” or “partially surrounding” means that the coating partially covers and partially surrounds the NSAID portion. In order to partially surround, the coating must contain at least a pair of selected points which can have a plain drawn between the points which intersects a section of the NSAID portion. Accordingly, a NSAID portion and a coating which are configured as a bilayer tablet would not meet this definition. Preferably, the plain intersects the NSAID portion for the coating to partially surround at least 5%, at least 10%, at least 25%, at least 50%, at least 75% or at least 95% of the NSAID portion. FIG. 4 depicts a coating which partially surrounds approximately 50% of the NSAID portion (the shaded portion). FIG. 4A depicts a coating which partially surrounds approximately 10% of the NSAID portion (the shaded portion).

The invention is further directed to the novel dosage forms and methods of preparation disclosed herein, without limitations with respect to the choice of drugs or class of drugs included in the dosage form.

### BRIEF DESCRIPTION OF THE DRAWINGS

FIG. 1 is a cross section of the embodiment of Example 1.

FIG. 1A is a top view of the embodiment of Example 1.

FIG. 1B is a side view of the embodiment of Example 1.

FIG. 2 is a cross section of an alternate embodiment of the invention.

FIG. 3 is a cross section of an alternate embodiment of the invention.

FIG. 4 is a cross section of an embodiment of the invention wherein the coating partially surrounds approximately 50% of the NSAID portion.

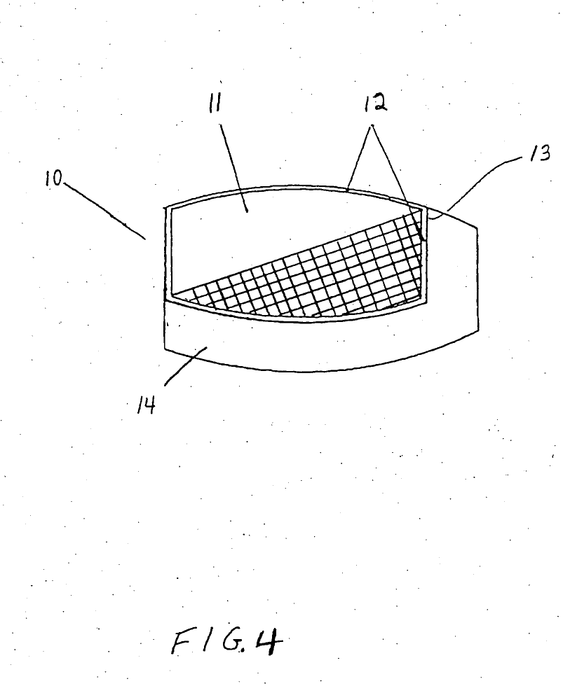

FIG. 4A is a cross section of an embodiment of the invention wherein the coating partially surrounds approximately 20% of the NSAID portion.

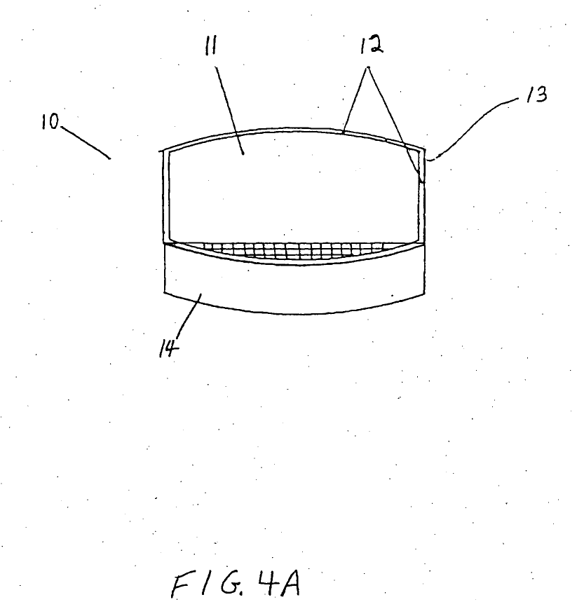

### DETAILED DESCRIPTION OF THE INVENTION

The term “NSAID,” as used herein, refers to any compound acting as a non-steroidal anti-inflammatory agent for the treatment of pain and/or inflammation. The treatment of pain includes all types of pain, including, but is not limited to, chronic pains, such as arthritis pain (e.g. pain associated with osteoarthritis and rheumatoid arthritis), neuropathic pain, and post-operative pain, chronic lower back pain, cluster headaches, herpes neuralgia, phantom limb pain, central pain, dental pain, neuropathic pain, opioid-resistant pain, visceral pain, surgical pain, bone injury pain, pain during labor and delivery, pain resulting from burns (including sunburn), post partum pain, migraine, angina pain, and genitourinary tract-related pain including cystitis, the term also refers to nociceptive pain or nociception.

The Merck Manual, 16th Edition, Merck Research Laboratories (1990) pp 1308-1309 provide well known examples of NSAIDs. The term NSAID includes, but is not limited to, the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen and tolmetin, including pharmaceutically acceptable salts, isomers and derivatives thereof, and combinations thereof. The salicylates may include acetylsalicylic acid, sodium acetylsalicylic acid, calcium acetylsalicylic acid, salicylic acid, and sodium salicylate. In certain preferred embodiments of the invention, the NSAID is diclofenac or at least one pharmaceutically acceptable salt thereof.

NSAIDs have been widely used in arthritis therapy for several years. The following references, hereby incorporated by reference, describe various NSAIDs suitable for use in the invention described herein, and processes for their manufacture: U.S. Pat. No. 3,558,690 to Sallmann and Pfister, (assigned to Ciba Geigy), issued 1971; U.S. Pat. No. 3,843,681 (assigned to American Home Products), issued 1974; U.S. Pat. No. 3,766,263 to Godfrey, (assigned to Reckitt and Colman) issued 1973; U.S. Pat. No. 3,845,215 to Godfrey (assigned to Reckitt and Colman) issued 1974; U.S. Pat. No. 3,600,437 to Marshall (assigned to Eli Lilly), issued 1971; U.S. Pat. No. 3,228,831 to Nicholson and Adams, (assigned to Boots Pure Drug), issued 1966; (U.S. Pat. No. 3,385,886 to Nicholson and Adams, (assigned to Boots Pure Drug) issued 1968; U.S. Pat. No. 3,161,654 to Shen, (assigned to Merck & Co.), issued 1964; U.S. Pat. No. 3,904,682 to Fried and Harrison, (assigned to Syntex), issued 1975; U.S. Pat. No. 4,009,197 to Fried and Harrison, (assigned to Syntex), issued 1977; U.S. Pat. No. 3,591,584 to Lombardino (assigned to Pfizer) issued 1971; U.S. Pat. No. 5,068,458 to Dales et al., (assigned to Beecham Group, PLC.), issued Nov. 26, 1991; U.S. Pat. No. 5,008,283 to Blackburn et al. (assigned to Pfizer, Inc.), issued Apr. 16, 1991; and U.S. Pat. No. 5,006,547 to Loose (assigned to Pfizer), issued Apr. 9, 1991. All of the above patents are hereby incorporated by reference.

When the antiulcerative compound of the present invention is a prostaglandin, the compound is preferably selected from the group consisting of misoprostol, PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{2α}, PGF_{3α}, and PGI₂, including pharmaceutically acceptable salts, isomers and derivatives thereof, and combinations thereof. In certain preferred embodiments, the antiulcerative compound is a prostaglandin, preferably misoprostol.

Prostaglandins have the tendency to be unstable when included in pharmaceutical dosage forms, therefore it is preferred that the prostaglandin contained in the dosage forms of the invention be stabilized by procedures known in the art, e.g., the procedures set forth in Derwent Abstract Nos. 90387A, 90386A, 90385A, 06805A and 32802W, which are hereby incorporated by reference. The stabilization of misoprostol is described in U.S. Pat. No. 4,301,146, which is hereby incorporated by reference.

Misoprostol is indicated for the prevention of NSAID (nonsteroidal anti-inflammatory drugs, including aspirin)-induced gastric ulcers in patients at high risk of complications from gastric ulcer, e.g., the elderly and patients with concomitant debilitating disease, as well as patients at high risk of developing gastric ulceration. It is recommended that misoprostol should be taken for the duration of NSAID therapy, which demonstrates the need for a combination product in accordance with the present invention.

The recommended adult oral dose of misoprostol for the prevention of NSAID-induced gastric ulcers is 200 mcg four times daily. If this dose cannot be tolerated, a dose of 100 mcg can be used.

When the antiulcerative compound of the present invention is an H₂ blocker, the compound is preferably selected from the group consisting of ranitidine, cimetidine, nizatidine, famotidine, pharmaceutically acceptable salts, isomers and derivatives thereof, single enantiomers thereof and combinations thereof.

When the antiulcerative compound of the present invention is a proton pump inhibitor, the compound is preferably selected from the group consisting of omeprazole, lansoprazole, rabeprazole, pantoprazole, leminoprazole, pharmaceutically acceptable salts, isomers and derivatives thereof, single enantiomers thereof and combinations thereof.

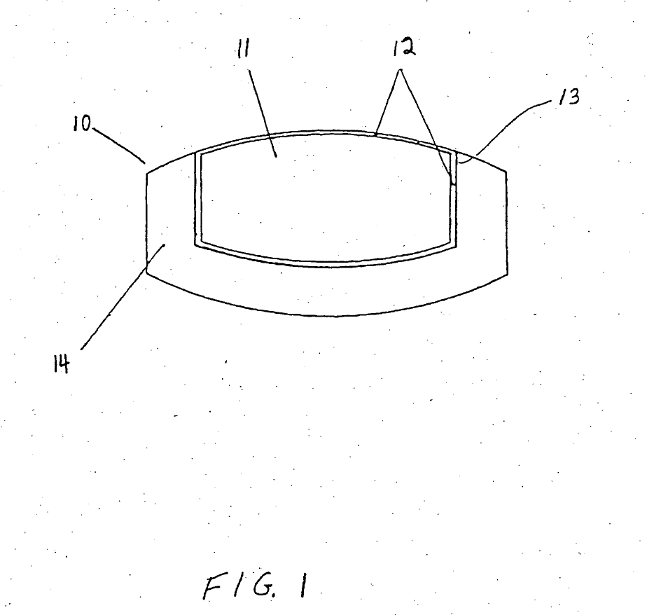

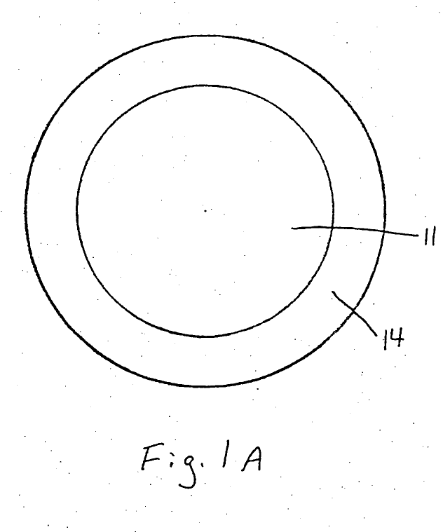

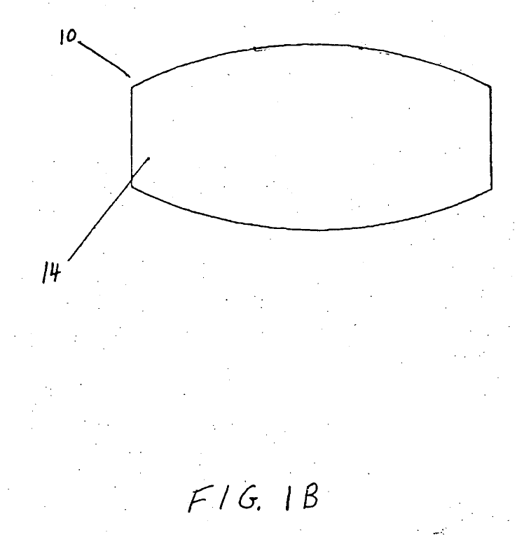

FIG. 1 represents a cross-section of an embodiment of a dosage form 10 of the present invention comprising a) an NSAID portion 11, having an optional enteric coating 12 and a seal coating 13; and b) an antiulcerative portion 14 that covers part but not all of the NSAID portion, partially surrounding the NSAID portion.

In this particular embodiment, the antiulcerative portion covers the surface of the diclofenac tablet on all surfaces except for the top surface. Accordingly, the NSAID is only visible from the top view of the formulation and the dosage form resembles a “bullseye” (e.g., a circle within a circle) as depicted in FIG. 1A. FIG. 1B represents a side-view of the dosage form of FIG. 1 wherein the NSAID is not visible.

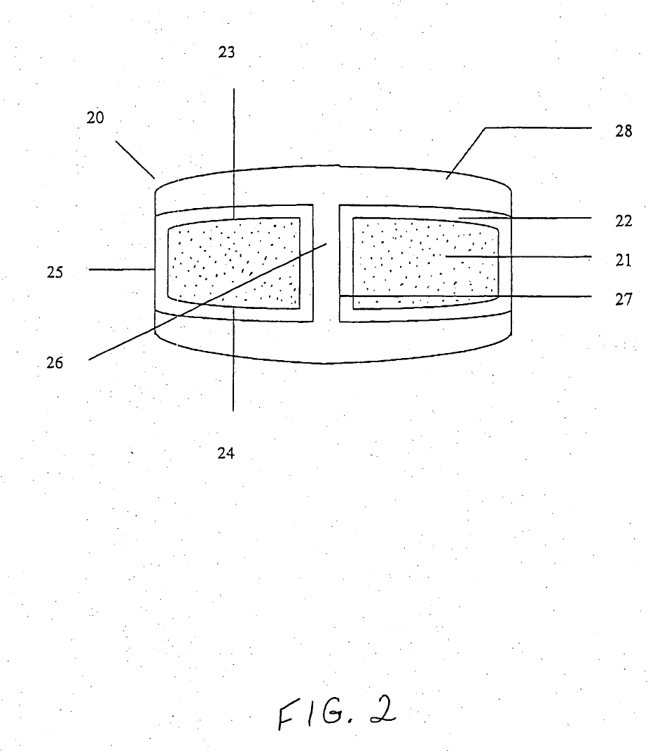

FIG. 2 represents a cross section of a dosage form 20 of the present invention comprising a) an NSAID portion 21 (with an optional enteric coating 22) comprising an NSAID, the NSAID portion having a top surface 23, a bottom surface 24, an outer circumferential surface 25 and an internal hole 26 extending from the top surface of the NSAID portion to the bottom surface of the NSAID portion and an inner circumferential surface 27; and b) a coating 28 comprising an antiulcerative compound coated onto the top surface and said bottom surface, the coating filling the internal hole to form an antiulcerative compound core in the dosage form. In this embodiment, the outer circumferential surface is not coated.

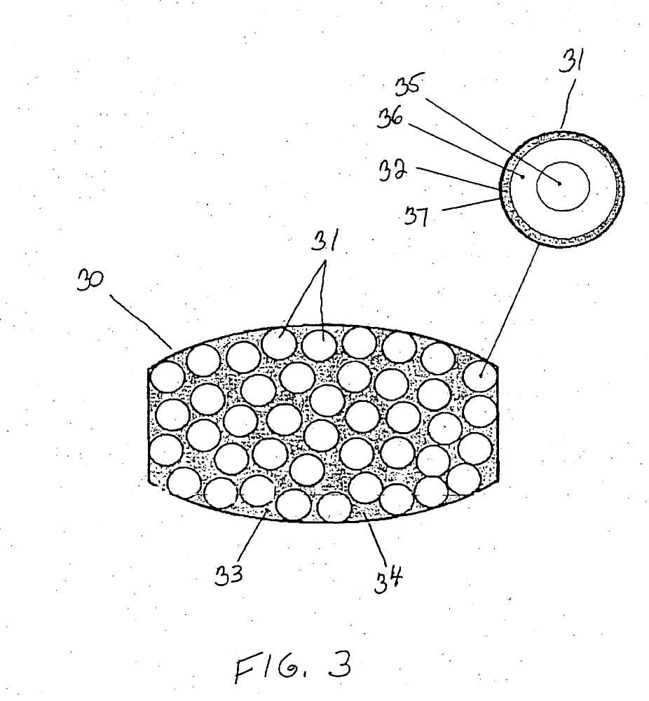

FIG. 3 represents an embodiment of a dosage form 30 of the present invention comprising a therapeutically effective amount of an NSAID contained in a plurality of multiparticulates 31 which are optionally coated with a delayed release layer 32. In such an embodiment, the NSAID multiparticulates are dispersed in a matrix 33 comprising a therapeutically effective amount of an antiulcerative compound 34. In FIG. 3, the multiparticulate comprises a sugar sphere 35 which is coated with the NSAID 36. The NSAID is optionally overcoated with an enteric coating 22 and is further optionally overcoated with a cushion or barrier coating 37.

In certain alternative embodiments, the internal hole does not extend through the NSAID portion, but provides for a cavity in the NSAID portion, which may be filled with the antiulcerative coating.

In certain embodiments, the present invention is directed to a method of preparing a solid oral dosage form comprising a) mixing an NSAID with one or more pharmaceutically acceptable excipients; b) compressing the mixture to form an NSAID portion; and c) applying a coating comprising an antiulcerative compound on the NSAID portion, wherein the coating partially surrounds the NSAID portion.

In certain embodiments, the mixture of NSAID with one or more pharmaceutically acceptable excipients is compressed to form an NSAID portion having a top surface, a bottom surface, an optional outer circumferential surface and an internal hole extending from the top surface of the NSAID portion to the bottom surface of the NSAID portion forming an inner circumferential surface, wherein the coating comprising an antiulcerative compound is applied onto the top surface and the bottom surface, and fills the internal hole to form an antiulcerative compound core in the dosage form. In certain embodiments, the coating can also be applied onto the outer circumferential surface. In certain alternate embodiments, the coating can be applied to completely surround the NSAID portion to provide to a hollow core (when the internal hole is not filled) or an antiulcerative core (when the internal hole is filled).

The multiparticulates can be immediate or controlled release matrices containing the NSAID or can consist of a plurality of pharmaceutically acceptable inert beads coated with the NSAID.

Preferably, the dosage form comprises a therapeutically effective amount of diclofenac coated onto a plurality of pharmaceutically acceptable inert beads and overcoated with an enteric coating, the enteric coated diclofenac beads being dispersed in a matrix comprising a therapeutically effective amount of misoprostol.

The inert beads can be coated with the diclofenac using known procedures, such as spray drying, spray congealing or powder layering.

In certain embodiments, the solid oral dosage forms comprising an NSAID and antiulcerative agent may further comprise other ingredients, including for example and without limitation, binders, surfactants, diluents, disintegrating agents, alkaline additives or other pharmaceutically acceptable ingredients, alone or in mixtures. Suitable diluents include, for example and without limitation, dicalcium phosphate, calcium sulfate, lactose, cellulose, kaolin, mannitol, sodium chloride, starches, powdered sugar, silicon dioxide, titanium oxide, alumina, talc, microcrystalline cellulose, mixtures thereof, and the like. Suitable binder materials include, for example and without limitation, starches (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, mixtures thereof, and the like), veegum, mixtures thereof, and the like. Suitable lubricants include, for example, magnesium stearate, calcium stearate, stearic acid, mixtures thereof, and the like. Disintegrants are for example starches, clays, celluloses, alginates, gums, crosslinked polymers, mixtures thereof, and the like. Suitable surfactants include pharmaceutically acceptable non-ionic, ionic and anionic surfactants. An example of a suitable surfactant is sodium lauryl sulfate. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. If desired, flavoring, coloring and/or sweetening agents may be added as well. Other optional components for incorporation into an oral formulation herein include, but are not limited to, preservatives, suspending agents, thickening agents, and the like.

In the present invention, an optional enteric coating layer may be applied onto the NSAID portion or multiparticulates using a suitable coating technique. The enteric coating layer material may be dispersed or dissolved in either water or in suitable organic solvents. As enteric coating layer polymers one or more, separately or in combination, of the following can be used: solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s), mixtures thereof, and the like. A useful enteric coating is an ethylacrylate methacrylic acid copolymer sold under the trademark Eudragit® by Rhom GmbH of Domstadt, Germany. Certain preferred enteric polymer coatings are for example Eudragit® L30D, L30D-55, HP50, HP55, L100, FS30D and S100.

The enteric coating layers preferably may contain effective amounts of pharmaceutically acceptable plasticizers to obtain the desired mechanical properties, such as flexibility and hardness of the enteric coating layers. Such plasticizers are, for example and without limitation, triacetin, citric acid esters, phthalic acid esters, dibutyl sebacate, cetyl alcohol, diethyl phthalate, triethyl citrate, polyethylene glycols, polysorbates or other plasticizers. The amount of plasticizer is optimized for the particular situation. The amount of plasticizer is usually above 10% by weight of the enteric coating layer polymer(s), preferably 15-50%, and more preferably 20-50%. Additives such as dispersants, colorants, pigments, polymers e.g. poly(ethylacrylate, methylmethacrylate), anti-tacking (e.g., talc) and anti-foaming agents may also be included into the enteric coating layer(s). Other compounds may be added to increase film thickness and to decrease diffusion of acidic gastric juices into the dosage form.

Overcoatings may be applied to the enteric coated NSAD portion, or multiparticulate as set forth above, e.g., by coating or layering procedures in suitable equipments such as coating pan, coating granulator or in a fluidized bed apparatus using water and/or organic solvents for the coating or layering process. Suitable overcoating materials include sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium and the like. Additives such as plasticizers, colorants, pigments, fillers, anti-tacking and anti-static agents, such as for instance magnesium stearate, titanium dioxide, talc and other additives may also be included in the over-coating layer(s).

The enteric coated tablets or multiparticulates are then coated (e.g., by compression coating) with the antiulcerative formulation.

Preferably the antiulcerative agent is mixed with suitable ingredients to form a free flowing antiulcerative agent granulation which can be incorporated with the NSAID portion, or multiparticulates by compression coating.

In certain embodiments as disclosed above, wherein the antiulcerative compound partially surrounds the NSAID, an NSAID tablet is precompressed and then is compression coated with the antiulcerative compound in order to cover all of the NSAID except for the top surface. Accordingly, the NSAID is only visible from the top view of the formulation and the dosage form resembles a “bullseye” (e.g., a circle within a circle).

In certain embodiments as disclosed above, when the NSAID formulation is in the form of a tablet with an internal hole, the compression coating preferably incorporates the antiulcerative coating on the top and bottom surfaces of the NSAID and completely fills the internal hole to provide the final dosage form with a shape of a conventional tablet. Preferably, the antiulcerative coating does not cover the outer circumferential surface of the NSAID tablet.

Such configurations as disclosed herein (with a portion of the NSAID exposed and not covered by the antiulcerative compound) may allow for dissolution of the NSAID prior to complete dissolution of the antiulcerative layer.

In this embodiment, the compaction of the antiulcerative agent in the NSAID portion internal hole to provide an antiulcerative inner core provides for the antiulcerative agent dissolution into biological fluids at a time after the antiulcerative coating on the top and bottom surfaces of the NSAID portion is dissolved. The dissolution of the antiulcerative coating in the NSAID portion internal hole will also dissolve. The antiulcerative coating in the NSAID portion internal hole may also dissolve at a reduced rate as compared to the antiulcerative coating on the top and bottom surfaces of the NSAID portion. This may be possible due to the reduced surface area of the antiulcerative coating in the NSAID portion interior hole as compared to the surface area of the antiulcerative agent on the top and bottom surfaces of the NSAID portion.

The final dosage form prepared in accordance with the invention are optionally covered with a film-forming agent(s) to obtain a smooth surface of the tablet and further enhance the stability of the tablet during packaging and transport. Such a tablet coating layer may further comprise additives like anti-tacking agents, colorants and pigments or other additives to obtain a tablet of good appearance.

In certain embodiments, the NSAID and the antiulcerative agent are both formulated to provide immediate release. In preferred embodiments, the immediate release NSAID formulation is enteric coated to provide a delayed release in order to avoid significant NSAID release in the gastric area and to provide dissolution of the NSAID in the intestine.

The immediate release formulations can be formulated with excipients known to those skilled in the art, with known procedures (e.g. direct compression). Such excipients and methods that may be used to formulate oral dosage forms are described in the Handbook of Pharmaceutical Excipients, American Pharmaceutical Association (1986), and Remington's Pharmaceutical Sciences, (Arthur Osol, editor), 1553-1593 (1980), both of which are hereby incorporated by reference.

In preferred embodiments, the NSAID is diclofenac or a pharmaceutically acceptable salt, isomer or derivative thereof and the antiulcerative agent is misoprostol or a pharmaceutically acceptable salt, isomer or derivative thereof.

When the diclofenac is formulated to provide immediate release, with or without the enteric coating, the NSAID portion or multiparticulates comprising the NSAID preferably contains up to about 100 mg of the drug. Specific amounts of diclofenac which are contemplated for use in the present invention can be e.g. 25 mg, 50 mg or 75 mg, although these amounts are not meant to be limiting. Immediate release formulations in accordance with the present invention may be administered B.I.D., T.I.D. or Q.I.D. for a total daily dose preferably not to exceed about 250 mg.

Misoprostol has been demonstrated to be effective in reducing the incidence of endoscopically diagnosed NSAID induced gastric ulcers at doses of 200 mcg B.I.D., T.I.D. or Q.I.D. as compared to a placebo. The T.I.D. and Q.I.D. regimen were therapeutically equivalent and the B.I.D. regimen was less effective than the T.I.D. and Q.I.D. regimens. The incidence of endoscopically induced duodenal ulcers was significantly reduced in all three dosing regimens. Accordingly, misoprostol is preferably included in the dosage form of the present invention in order to provide a total daily dose not to exceed about 800 mcg, preferably from about 400 mcg to about 800 mcg daily, more preferably from about 600 mcg to about 800 mcg daily. Preferably, a dosage form of the present invention contains misoprostol from about 50 mcg to about 200 mcg, based on the variant dosing of the formulation. In most preferred embodiments, the dosage form includes 200 mcg misoprostol.

The NSAID of the present invention (e.g., diclofenac) can also formulated in a sustained release form in order to reduce the number of NSAID doses per day, thereby improving patient compliance and efficacy. The sustained release dosage form may optionally include a sustained released carrier which is incorporated into a matrix along with an effective amount of NSAID to provide a controlled release of the NSAID for at least about 24 hours. The dosage form can optionally be in multiparticulate form. In such an embodiment, it may be necessary to provide the prostaglandin in an effective amount as a sustained release formulation to provide a gastrointestinal protective effect for a corresponding amount of time (e.g. 24 hours).

The retardant material which may be included in the NSAID portion, or multiparticulates or the antiulcerative coating/matrix can include one or more pharmaceutically acceptable hydrophobic materials and/or hydrophilic materials which are capable of imparting controlled release of the active agent in accordance with the present invention. In the embodiment wherein the NSAID is in a tablet form comprising an NSAID portion and an antiulcerative portion, and both drugs are in sustained release form, it is preferable that the antiulcerative coating not cover the outer circumferential surface of the NSAID portion in order to allow for the initiation of NSAID dissolution through this surface.

The hydrophobic material is preferably selected from the group consisting of alkylcelluloses (e.g., ethylcellulose), acrylic and methacrylic acid polymers and copolymers, hydrogenated castor oil, hydrogenated vegetable oil, gums, protein derived materials, aliphatic alcohols, glycerol monostearate, stearic acid, canauba wax or mixtures thereof.

In certain embodiments of the present invention, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. In other embodiments, the hydrophobic material is selected from materials such as alkylcelluloses, e.g., methylcellulose or ethylcellulose. In other embodiments, the hydrophobic material is an aliphatic alcohol, e.g., lauryl alcohol, myristyl alcohol, or stearyl alcohol.

An example of a suitable retardant material having hydrophilic properties is a hydroxyalkylcellulose, e.g., hydroxypropylmethylcellulose.

This list is not meant to be exclusive, and any pharmaceutically acceptable hydrophobic material and/or hydrophilic material which are capable of imparting controlled release of the active agent may be used in accordance with the present invention.

Prior to compressing the NSAID portion or multiparticulates with the antiulcerative portion, the NSAID portion or NSAID multiparticulates (with or without an enteric coating) can be coated with a pharmaceutically acceptable film-coating or cushion coating, e.g., for stability purposes (e.g., coated with a moisture barrier), etc. For example, the NSAID portion or multiparticulates may be overcoated with a film coating, preferably containing a pigment and a barrier agent, such as hydroxypropylmethylcellulose and/or a polymethylmethacrylate. An example of a suitable material which may be used for such a hydrophilic coating is hydroxypropylmethylcellulose (e.g., Opadry®, commercially available from Colorcon, West Point, Pa.). Any pharmaceutically acceptable manner known to those skilled in the art may be used to apply the coatings. For example, the coating may be applied using a coating pan or a fluidized bed. An organic, aqueous or a mixture of an organic and aqueous solvent is used for the hydrophobic polymer or enteric coating. Examples of suitable organic solvents are, e.g., isopropyl alcohol, ethanol, and the like, with or without water. Aqueous solvents are preferred for the overcoating procedures.

In preferred embodiments, the antiulcerative compound is coated onto the NSAID portion by compression coating which provides for the antiulcerative coating to be compressed in the interior hole of the NSAID portion to form an antiulcerative core in the NSAID portion.

In an alternate embodiment, the antiulcerative can be spray dried onto the surface of the tablet using any spray technique known to those skilled in the art. This coating can also be applied using a coating pan or a fluidized bed using an organic, aqueous or a mixture of an organic and aqueous solvent for the antiulcerative agent. Aqueous solvents are preferred for the spray coating. In such an embodiment, the final dosage form can retain the interior hole of the tablet.

In certain embodiments, when the coating comprises an unstable prostaglandin such as misoprostol, the coating further comprises a polymer selected from the group consisting of hydroxypropylmethylcellulose, polyvinylpyrolidone and combinations thereof to stabilize the drug. Preferably, the polymer is present in an amount from about 50 to about 500 parts per part of the drug. This polymer can also be used in the embodiment wherein diclofenac multiparticulates are compressed with a misoprostol matrix.

### DETAILED DESCRIPTION OF CERTAIN PREFERRED EMBODIMENTS

The following examples illustrate various aspects of the present invention. They are not to be construed to limit the claims in any manner whatsoever.

### EXAMPLE 1

### Preparation of Diclofenac Sodium Tablet with Misoprostol Immediate Release Layer

The formulation of Example 1 was prepared by forming a granulation of diclofenac sodium, lactose, Avicel, povidone, crospovidone, and magnesium stearate and compressing the granulation into a tablet. A delayed-release coating comprising Eudragit L30D, talc, and triethyl citrate was then applied onto the diclofenac tablet to produce delayed-release coated diclofenac tablets. The delayed-release tablets were then seal coated with a mixture of hydroxypropylmethylcellulose and polyethyleneglycol. Thereafter the seal coated delayed release tablets were compression coated with a mixture of misoprostol HPMC dispersion, Avicel, crospovidone XL and hydrogenated vegetable (castor) oil. The ingredients of the final dosage form are set forth in Table 1:

TABLE 1
Diclofenac Sodium Delayed Release Tablets

| Ingredients | Percent (%) |
|---|---|
| Tablet | |
| Diclofenac Sodium | 55.143 |
| Lactose | 12.725 |
| Avicel | 12.725 |
| Povidone | 4.242 |
| Crospovidone | 3.572 |
| Mg. Stearate | 0.893 |
| Delayed Release Coating | |
| L30D | 3.56 |
| Talc | 1.78 |
| TEC | 0.356 |
| Overcoat (seal coat) | |
| HPMC | 4.0 |
| PEG | 1.0 |
| Misoprostol Immediate Release Layer | |
| Misoprostol HPMC dispersion | 5.05 |
| Avicel | 88.95 |
| Crospovidone XL | 5.0 |
| Hydrogenated vegetable (castor) oil | 1.0 |
| Placebo Layer | |
| HPMC | 5.05 |
| Avicel | 88.95 |
| Crospovidone XL | 5.0 |
| Hydrogenated vegetable (castor) oil | 1.0 |

In the final formulation, the compression coating of the misoprostol covered the surface of the diclofenac tablet on all surfaces except for the top surface. Accordingly, the NSAID is only visible from the top view of the formulation and the dosage form resembles a “bullseye” (e.g., a circle within a circle) as depicted in FIG. 1A.

### EXAMPLE 2

### Preparation of Diclofenac Sodium/Misoprostol Tablets, 50 mg/200 mcg

The formulation of Example 2 was prepared by coating a plurality of inert sugar cores with a diclofenac sodium containing layer. A delayed-release coating comprising cellulose acetate phthalate and diethyl phthalate is then applied onto the diclofenac-containing bead to produce delayed-release coated diclofenac beads. An optional overcoat comprising povidone K-30 and talc is then applied to the delayed-release coated beads. A plurality of beads are then blended with misoprostol, hydroxypropylmethylcellulose, Avicel, crospovidone and glyceral monostearate, and compressed into tablets. The ingredients of the final dosage form are in the ratio as set forth in Table 2:

TABLE 2

| Ingredients | Ratio |
|---|---|
| ACTIVE PELLETS | |
| Sugar spheres | 26.7 |
| Diclofenac Sodium | 15.0 |
| Povidone K-30 | 1.0 |
| DELAYED-RELEASE (ENTERIC) COATING | |
| Cellulose Acetate Phthalate | 4 |
| Diethyl Phthalate | 1 |
| OVERCOAT (OPTIONAL) | |
| Povidone K-30 | 1 |
| Talc | 1 |
| CUSHION OR TABLET MATRIX | |
| HPMC (E5) + Misoprostol | 1 |
| Avicel | 10 |
| Crospovidone | 1.25 |
| Glyceryl monostearate | 0.25 |

### EXAMPLE 3

### Preparation of Diclofenac Sodium/Misoprostol Tablets, 50 mg/200 mcg

Example 3 is formulated by preparing a diclofenac sodium granulation and compressing unit dosage forms in a “donut” shape (i.e. having an inner cavity). Each tablet has the following composition in Table 3 below:

TABLE 3

| Ingredient | Amount (mg) |
|---|---|
| Diclofenac Sodium | 50.0 |
| lactose (monohydrate) | 13.0 |
| microcrystalline cellulose | 12.9 |
| cornstarch | 8.4 |
| povidone K-30 | 4.8 |
| magnesium stearate | 0.9 |

The diclofenac sodium 50 mg tablets are then enteric coated by known procedures with a combination of Hydroxypropyl Methylcellulose Phthalate 50 NF, Talc USP and Cetyl Alcohol.

Each enteric coated tablet is then coated by compression coating with 200 mcg misoprostol and a sufficient amount of excipient to fill the inner cavity and coat the top and bottom surface of the tablet, excluding the outer circumferential surface.

1. A solid oral dosage form comprising:
a first potion comprising a therapeutically effective amount of an NSAID; and
a coating comprising a therapeutically effective amount of an antiulcerative compound;
said coating at least partially surrounding said first NSAID portion.

2. The solid oral dosage form of claim 1, further comprising an enteric coating on said NSAID portion.

3. The solid oral dosage form of claim 2, further comprising a layer between said enteric coating and said coating comprising said antiulcerative compound.

4. The solid oral dosage form of claim 1, which is a tablet.

5. The solid oral dosage form of claim 1, wherein said NSAID is selected from the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts, isomers and derivatives thereof, and combinations thereof.

6. The solid oral dosage form of claim 1, wherein said NSAID is diclofenac or a pharmaceutically acceptable salt, isomer or derivative thereof.

7. The solid oral dosage form of claim 1, wherein said antiulcerative compound is selected from the group consisting of a prostaglandin, an H₂ blocker, a proton pump inhibitor and combinations thereof.

8. The solid oral dosage form of claim 1, wherein said antiulcerative agent is a prostaglandin selected from the group consisting of misoprostol, PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{2α}, PGF_{3α}, PGI₂, pharmaceutically acceptable salts, isomers and derivatives thereof, and combinations thereof.

9. The solid oral dosage form of claim 1, wherein said antiulcerative agent is misoprostol.

10. The solid oral dosage form of claim 1, wherein said antiulcerative agent is an H₂ blocker selected from the group consisting of ranitidine, cimetidine, nizatidine, famotidine, pharmaceutically acceptable salts thereof, single enantiomers thereof, isomers thereof, derivatives thereof and combinations thereof.

11. The solid oral dosage form of claim 1, wherein said antiulcerative agent is a proton pump inhibitor selected from the group consisting of omeprazole, lansoprazole, rabeprazole, pantoprazole, leminoprazole, single enantiomers thereof, pharmaceutically acceptable salts thereof isomers thereof, derivatives thereof and combinations thereof.

12. The solid oral dosage form of claim 1, wherein said NSAID is diclofenac or a pharmaceutically acceptable salt isomer or derivative thereof, and said antiulcerative is misoprostol.

13. The solid oral dosage form of claim 1, wherein said coating comprises a polymer selected from the group consisting of hydroxypropylmethylcellulose, hydroxypropylcellulose, polyvinylpyrolidone, propylene glycol and combinations thereof.

14-16. (canceled)

17. A method of treating pain or inflammation in a patient comprising administering to said patient the solid oral dosage form of claim 1.

18. A solid oral dosage form comprising:
an NSAID portion having at least one internal hole extending through said portion;
a coating portion comprising an antiulcerative compound at least partially filling the internal hole of the NSAID portion.

19. The solid oral dosage form of claim 18, wherein said coating is further coated onto at least a portion of the outer surface of the NSAID portion.

20-29. (canceled)

30. The dosage form of claim 1, wherein said NSAID portion comprises a matrix comprising said NSAID and a retardant material.

31. The solid dosage form of claim 30, wherein said retardant material is an aliphatic alcohol.

32. The solid dosage form of claim 31, wherein said aliphatic alcohol is stearyl alcohol.

33-34. (canceled)

35. A method of preparing a solid oral dosage form comprising:
a) mixing an NSAID with pharmaceutically acceptable excipients;
b) compressing the mixture to form an NSAID portion; and
c) applying a coating comprising an antiulcerative compound on the NSAID portion, wherein said coating at least partially surrounds said NSAID portion.

36. The method of claim 35, wherein said NSAID portion has a top surface, a bottom surface, and an internal hole extending from the top surface of the NSAID portion to the bottom surface of the NSAID portion; and wherein said coating is applied onto the top surface and the bottom surface of said NSAID portion and fills the internal hole forming an antiulcerative compound core in the dosage form.

37. (canceled)

38. The method of claim 35 wherein the coating is applied by compression coating.

39. A solid oral dosage form comprising a therapeutically effective amount of an NSAID contained in a plurality of multiparticulates; the NSAID multiparticulates dispersed in a matrix comprising a therapeutically effective amount of an antiulcerative compound.

40-48. (canceled)

49. A method of preparing a solid oral dosage form comprising
d) coating an NSAID onto a plurality of pharmaceutically acceptable inert beads;
e) overcoating the beads with a delayed release coating;
f) blending the beads with a matrix material comprising an antiulcerative compound and at least one pharmaceutically acceptable excipient; and
g) compressing a sufficient amount of the blend into a dosage form to contain a therapeutically effective amount of the NSAID and the antiulcerative compound, wherein said NSAID beads are dispersed in the matrix material.

50. (canceled)
